(19) United States
(12) Patent Application Publication
(10) Pub. No.: US 2024/0373863 A1
(43) Pub. Date: Nov. 14, 2024

(54) METHODS FOR MAKING FERMENTED FOOD AND BEVERAGE PRODUCTS USING GY7B YEAST

(71) Applicant: Saint Joseph's University, Philadelphia, PA (US)

(72) Inventor: Matthew J. Farber, Philadelphia, PA (US)

(73) Assignee: Saint Joseph's University, Philadelphia, PA (US)

(21) Appl. No.: 18/559,090

(22) PCT Filed: May 5, 2022
(86) PCT No.: PCT/US2022/027756

§ 371 Date: Nov. 6, 2023

Related U.S. Application Data
(60) Provisional application No. 63/185,698, filed on May 7, 2021.

(51) Int. Cl. A21D 8/04; A23L 2/38; A23L 2/82; A23L 2/84; C12C 11/11; C12C 12/00

(52) U.S. Cl. CPC A21D 8/047; A21D 8/045; A23L 2/382; A23L 2/82; A23L 2/84; C12C 11/11; C12C 12/006

(57) ABSTRACT

The present disclosure relates, in part, to methods of producing fermented food and beverage products, wherein the fermentation comprises yeast strain GY7B. In another aspect, the present disclosure provides a method promoting flocculation of at least one yeast strain utilizing yeast strain GY7B.

### CROSS-REFERENCE TO RELATED APPLICATIONS

The present application claims priority under 35 U.S.C. § 119 (e) to U.S. Provisional Patent Application No. 63/185,698, filed May 7, 2021, which is incorporated herein by reference in its entirety.

### DEPOSIT STATEMENT

The GY7B yeast strain was deposited, in accordance with the Budapest Treaty, with the American Type Culture Collection (ATCC®) on Jul. 19, 2018, under Accession Number PTA-125167. In accordance with 37 CFR § 1.808, the depositors assure that all restrictions imposed on the availability to the public of the deposited materials will be irrevocably removed upon the granting of a patent.

### BACKGROUND

It is estimated that there are 5.1 million different species of yeast, yet only 1-2% have been characterized and described. Different strains of yeast have varying properties, including fermentation performance and flavor. Examples of commercially useful yeast strain categories include “baker's yeast” (which is a leavening agent) and “brewer's yeast” (which is used for alcoholic fermentation processes). It should be noted that, within each category, specific strains can produce distinct metabolic byproducts, which alter the properties of the food products in which they are incorporated.

Flavor is among the properties of food and beverage products that are influenced by the particular strains of yeast and/or bacteria during the fermentation process. The primary method whereby a sour taste is imparted in a fermented food or beverage, such as beer or sourdough, involves the utilization of lactic acid bacteria (LAB), primarily Lactobacillus or Pediococcus, in the fermentation process. Such methods involve the intentional addition of LAB or the spontaneous inclusion of LAB in the culture, as demonstrated in the creation of traditional sourdough starters. In the latter process, the baker relies upon the natural yeast and bacteria present in the flour to flavor (with acidity) and leaven the bread.

There is thus a need in the art for methods of imparting sour flavor in various fermented foods and beverages without the use of LAB. The present disclosure addresses this need.

### SUMMARY

In some embodiments, the instant specification is directed to, among others, methods of producing sourdough bread, methods of producing a non-alcoholic fermented beverage, methods of promoting flocculation of yeast strain, and methods of producing an alcoholic fermented beverage.

### Method of Producing Sourdough Bread

In some embodiments, the instant specification is directed to a method of producing sourdough bread.

In some embodiments, the method comprises baking fermented dough, wherein the fermented dough comprises yeast strain GY7B.

In some embodiments, the dough is fermented in the absence of any acid producing bacteria.

In some embodiments, the fermented dough is prepared from a starter culture.

In some embodiments, the starter culture comprises flour, water, and at least one yeast strain.

In some embodiments, the at least one yeast strain is yeast strain GY7B.

In some embodiments, the at least one yeast strain is selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus, Hansensiaspora uvarum, Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae, Wickerhamomyces subpelliculosus, Wickerhamomyces anomalus, Zygosaccharomyces rouxii, Zygosaccharomyces bailii, Zygosaccharomyces fermentati, Zygosaccharomyces florentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

In some embodiments, the fermented dough comprises at least one supplement.

In some embodiments, the at least one supplement is selected from the group consisting of sugar and yeast extract.

In some embodiments, the sugar is selected from the group consisting of glucose, sucrose, fructose, and maltose.

In some embodiments, the yeast extract comprises at least one selected from the group consisting of zinc salts, calcium salts, magnesium salts, lipids, peptides, and amino acids.

Method of producing a non-alcoholic fermented beverage

In some embodiments, the instant specification is directed to a method of producing a non-alcoholic fermented beverage.

In some embodiments, the method comprises fermenting a substrate in the presence of yeast strain GY7B.

In some embodiments, the substrate is fermented in the absence of any acid producing bacteria.

In some embodiments, the substrate is fermented in the presence of at least one additional yeast strain selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus, Hansensiaspora uvarum, Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae, Wickerhamomyces subpelliculosus, Wickerhamomyces anomalus, Zygosaccharomyces rouxii, Zygosaccharomyces bailii, Zygosaccharomyces fermentati, Zygosaccharomyces florentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

In some embodiments, the non-alcoholic fermented beverage has a low pH.

In some embodiments, the non-alcoholic fermented beverage has an amount of ethanol ranging from about 0.05% to about 0.5% (v/v).

In some embodiments, the substrate is at least one selected from the group consisting of malt, must, honey, and tea.

In some embodiments, the fermentation comprises at least one supplement.

In some embodiments, the at least one supplement is selected from the group consisting of a fruit, processed fruit derivative, and an enzyme.

In some embodiments, the fruit or processed fruit derivative is a sugar.

In some embodiments, the sugar is glucose.

In some embodiments, the glucose is added in an amount ranging from about 1% to about 5% substrate to total fermentation volume (w/v).

In some embodiments, the enzyme is selected from the group consisting of a β-glucosidase and amylase.

In some embodiments, the supplement is present during early fermentation.

In some embodiments, the fermentation occurs with a yeast cell count of about 1.0×10⁶ cells/mL.

In some embodiments, the fermentation occurs with a yeast cell count ranging from about 0.5 g/L to about 1.5 g/L.

In some embodiments, the GY7B yeast strain is removed and/or inactivated.

In some embodiments, the removal comprises at least one of centrifugation, filtration, and physical separation.

In some embodiments, the inactivation comprises at least one of low temperature exposure, pasteurization, and chemical inhibitor.

In some embodiments, the chemical inhibitor is potassium sorbate.

### Method of Promoting Flocculation of Yeast Strain

In some embodiments, the instant specification is directed to a method of promoting flocculation of at least one yeast strain.

In some embodiments, the method comprises adding yeast strain GY7B to a fermentation vessel comprising the at least one yeast strain before and/or after fermentation of a substrate.

In some embodiments, the substrate comprises a malt derived from at least one grain selected from the group consisting of barley, wheat, corn, rye, rice, oats, sorghum, millet, buckwheat, quinoa, and teff.

In some embodiments, the at least one yeast strain is yeast strain GY7B.

In some embodiments, the at least one yeast strain is selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus, Hansensiaspora uvarum, Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae, Wickerhamomyces subpelliculosus, Wickerhamomyces anomalus, Zygosaccharomyces rouxii, Zygosaccharomyces bailii, Zygosaccharomyces fermentati, Zygosaccharomyces florentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

### Method of Producing Alcoholic Fermented Beverage

In some embodiments, the instant specification is directed to a method of producing an alcoholic fermented beverage.

In some embodiments, the method comprises fermenting a substrate in the presence of yeast strain GY7B.

In some embodiments, the fermented alcoholic beverage is selected from the group consisting of wine, cider, seltzer, and mead.

In some embodiments, the alcoholic fermented beverage is produced without both sequential fermentation and co-fermentation.

In some embodiments, the alcoholic fermented beverage has a low pH.

In some embodiments, the pH has a range of about pH 2.0 to about pH 4.0.

In some embodiments, the substrate is fermented in the absence of an acid producing bacteria.

In some embodiments, the fermentation of the substrate occurs in the presence of at least one additional yeast strains selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus, Hansensiaspora uvarum, Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae, Wickerhamomyces subpelliculosus, Wickerhamomyces anomalus, Zygosaccharomyces rouxii, Zygosaccharomyces bailii, Zygosaccharomyces fermentati, Zygosaccharomyces florentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting: information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

As used herein, each of the following terms has the meaning associated with it in this section.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, exemplary methods and materials are described.

Generally, the nomenclature used herein and the laboratory procedures in yeast culturing and beer brewing are those well-known and commonly employed in the art.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The term “flocculant” or “flocculation” as used herein refers to a substance that promotes the aggregation and/or precipitation of particles in a suspension. In certain embodiments, the particles in the suspension comprise microorganisms including, but not limited to, yeast cells. In certain embodiments, the particles in suspension comprise haze-forming macromolecules including, but not limited to, protein and polyphenols.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “leavening agent” as used herein refers to a composition which causes an expansion of a malleable paste to a foam by the release of a gas within the malleable paste. Non-limiting examples of malleable pastes include dough and batter. In certain embodiments, the leavening agent is yeast.

The term “non-alcoholic” as used herein refers to a drink or beverage having an alcohol (i.e. ethanol) by volume (ABV) content which is less than or equal to 0.5% (v/v).

The term “starter culture” or “fermentation starter” as used herein refers to a microbiological culture, which typically comprises a cultivation medium, including but not limited to grains, seeds, or nutrient liquids, which have been colonized by microorganisms. The variety and abundance of particular microorganisms comprising a starter culture may vary. Non-limiting examples of microorganisms comprising a starter culture include bacteria and yeasts. The starter is used to assist in the fermentation process used to prepare various food and beverage products.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substrate” as used herein refers to the carbohydrate source which is consumed a microorganism during fermentation. The substrate may comprise a simple sugar (e.g. mono-and/or disaccharide), a complex carbohydrate or polysaccharide (e.g. grain), or any combination thereof.

The terms “wort” and “must” as used herein to refer to an aqueous mixture or suspension comprising the components necessary for the production of a fermented beverage prior to inoculation with a fermentation agent (i.e. yeast). Thus, the “wort” or “must” comprises the carbohydrate containing fermentation substrate (e.g. sugar or grain) and any of a number of additives included for the production of the intended fermented beverage.

### GY7B Yeast Strain

An exemplary yeast strain of the disclosure is the GY7B yeast strain, which is described in International Application Publication No. WO 2019/018803, published Jan. 24, 2019, all of which is incorporated herein in its entirety by reference. Under the terms of the Budapest Treaty on the International Recognition of the Deposit of Microorganisms for the Purpose of Patent Procedure, deposit of the yeast strain is being made with the American Type Culture Collection (ATCC) of Rockville, Md., USA.

Applicant's assignee, University of the Sciences, represents that the ATCC is a depository afforded permanence of the deposit and ready accessibility thereto by the public if a patent is granted. All restrictions on the availability to the public of the material so deposited will be irrevocably removed upon granting of a patent. The material will be readily available during the pendency of the patent application to one determined by the Commissioner to be entitled thereto under 37 C.F.R. § 1.14 and 35 U.S.C. § 122. The deposited material will be maintained with all the care necessary to keep it viable and uncontaminated for a period of at least five years after the most recent request for the furnishing of a sample of the deposited material, and in any case, for a period of at least thirty (30) years after the date of the deposit or for the enforceable life of the patent, whichever period is longer. Applicants' assignee acknowledges its duty to replace the deposit should the depository be unable to furnish a sample when requested due to the condition of the deposit.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Methods

In one aspect, the present disclosure provides a method of producing sourdough bread.

In certain embodiments, the method comprises baking fermented dough.

In certain embodiments, the fermented dough comprises yeast strain GY7B.

In certain embodiments, the dough is fermented in the absence of any acid producing bacteria.

In certain embodiments, the fermented dough is prepared from a starter culture.

In certain embodiments, the starter culture comprises flour, water, and at least one yeast strain.

In certain embodiments, the at least one yeast strain is yeast strain GY7B.

In certain embodiments, the at least one yeast strain selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus Hansensiaspora uvarum Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae Wickerhamomyces subpelliculosus, Wickerhamomyces anomalus, Zygosaccharomyces rouxii, Zygosaccharomyces bailii, Zygosaccharomyces fermentati, Zygosaccharomycesflorentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

In certain embodiments, the fermented dough comprises at least one supplement.

In certain embodiments, the at least one supplement is selected from the group consisting of sugar and yeast extract.

In certain embodiments, the sugar is selected from the group consisting of glucose, sucrose, and maltose.

In certain embodiments, the yeast extract comprises at least one selected from the group consisting of zinc salts, calcium salts, magnesium salts, lipids, peptides, and amino acids.

In certain embodiments, only one strain of yeast is used as a souring agent in a starter culture and as a leavening agent during bread production.

In certain embodiments, the only one strain of yeast used as both a souring agent and as a leavening agent is GY7B.

In another aspect, the present disclosure provides a method of producing a non-alcoholic fermented beverage.

In certain embodiments, the method comprises fermenting a substrate in the presence of yeast strain GY7B.

In certain embodiments, the substrate is fermented in the absence of any acid producing bacteria.

In certain embodiments, the substrate is fermented in the presence of at least one additional yeast strain selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus Hansensiaspora uvarum Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae Wickerhamomyces subpelliculosus, Wickerhamomyces anomalus, Zygosaccharomyces rouxii, Zygosaccharomyces bailii, Zygosaccharomyces fermentati, Zygosaccharomycesflorentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

In certain embodiments, the non-alcoholic fermented beverage has a low pH. In certain embodiments, the pH is selected from the group consisting of about pH 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, and about pH 4.0.

In certain embodiments, the non-alcoholic fermented beverage has an amount of ethanol ranging from about 0.05% to about 0.5% (v/v).

In certain embodiments, the substrate is at least one selected from the group consisting of malt and must.

In certain embodiments, the fermentation comprises at least one supplement. In certain embodiments, the at least one supplement is selected from the group consisting of a fruit, processed fruit derivative, and an enzyme. In certain embodiments, fruit or processed derivative thereof is a sugar. In certain embodiments, the sugar is glucose. In certain embodiments, the glucose is added in an amount ranging from about 1% to about 5% substrate to total fermentation volume (w/v). In certain embodiments, the enzyme is selected from the group consisting of a B-glucosidase and amylase. In certain embodiments, the supplement is present during early fermentation.

In certain embodiments, the fermentation occurs with a yeast cell count of about 1.0×10⁶ cells/mL. In certain embodiments, the fermentation occurs with a yeast cell count ranging from about 0.5 g/L to about 1.5 g/L.

In certain embodiments, the GY7B yeast strain is removed and/or inactivated. In certain embodiments, the removal comprises at least one of centrifugation, filtration, and physical separation. In certain embodiments, the inactivation comprises at least one of low temperature exposure, pasteurization, and chemical inhibitors. In certain embodiments, the chemical inhibitor is potassium sorbate. In certain embodiments, the inactivation occurs before lactic acid fermentation. In certain embodiments, the inactivation occurs after lactic acid fermentation. In certain embodiments, the inactivation occurs before alcoholic fermentation. In certain embodiments, the inactivation occurs after alcoholic fermentation.

In another aspect, the present disclosure provides a method of promoting flocculation of at least one yeast strain.

In certain embodiments, the method comprises adding yeast strain GY7B to a fermentation vessel comprising the at least one yeast strain before and/or after fermentation of a substrate.

In certain embodiments, the substrate comprises a malt derived from at least one grain selected from the group consisting of barley, wheat, corn, rye, rice, oats, sorghum, millet, buckwheat, quinoa, and teff.

In certain embodiments, the at least one yeast strain is yeast strain GY7B.

In certain embodiments, the at least one yeast strain is selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus Hansensiaspora uvarum Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae Wickerhamomyces subpelliculosus, Wickerhamomyces anomalus, Zygosaccharomyces rouxii, Zygosaccharomyces bailii, Zygosaccharomyces fermentati, Zygosaccharomycesflorentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

In another aspect, the present disclosure provides a method of producing an alcoholic fermented beverage.

In certain embodiments, the method comprises fermenting a substrate in the presence of yeast strain GY7B.

In certain embodiments, the fermented alcoholic beverage is selected from the group consisting of wine, cider, and mead.

In certain embodiments, the alcoholic fermented beverage has a low pH.

In certain embodiments, the substrate is fermented in the absence of any acid producing bacteria.

In certain embodiments, the fermentation of the substrate occurs in the presence of at least one additional yeast strains selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus Hansensiaspora uvarum Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae Wickerhamomyces subpelliculosus, Wickerhamomyces anomalus, Zygosaccharomyces rouxii, Zygosaccharomyces bailii, Zygosaccharomyces fermentati, Zygosaccharomycesflorentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

In certain embodiments, the alcoholic fermented beverage is wine.

In certain embodiments, the wine is produced without both sequential fermentation and co-fermentation.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Examples

Various embodiments of the present application can be better understood by reference to the following Examples which are offered by way of illustration. The scope of the present application is not limited to the Examples given herein.

### Example 1

### Sourdough Bread

In certain embodiments, sourdough bread is prepared from a starter culture. In certain embodiments, the starter culture comprises flour, water, and yeast. In certain embodiments, the starter culture comprises flour, water, yeast, and lactic acid bacteria (LAB). In certain embodiments, the yeast comprises endogenous yeast. In certain embodiments, the yeast comprises endogenous bacteria. In certain embodiments, the yeast comprises GY7B. In certain embodiments, the starter culture further comprises at least one bacterial strain. In certain embodiments, the sourdough bread is prepared without a starter culture. In certain embodiments, the sourdough bread is prepared with GY7B which provides lactic acid (i.e souring agent) and acts as a leavening agent.

In certain embodiments, the starter culture may be fed after a period of time with additional water and/or flour. In certain embodiments, the starter culture undergoes fermentation. In certain embodiments, the fermentation occurs in a vessel which is at least partially sealed. In certain embodiments, the vessel is completely sealed.

In certain embodiments, dough comprises flour, water, and at least one yeast strain. In certain embodiments, the dough further comprises a starter culture. In certain embodiments, the dough is prepared without GY7B. In certain embodiments, GY7B is added directly to the dough. In certain embodiments, the dough was prepared with a starter culture comprising GY7B. In certain embodiments, the dough is prepared with a starter culture comprising GY7B and GY7B was added directly to the dough.

In certain embodiments, supplements may be added to at least one of the starter culture and the dough.

In certain embodiments, the dough undergoes fermentation. In certain embodiments, the fermentation occurs in a sealed vessel. In certain embodiments, the dough undergoes fermentation until a sufficient increase in volume of the dough is observed. In certain embodiments, the fermentation of the dough produces risen dough.

In certain embodiments, the risen dough is baked at a suitable temperature for a suitable amount of time.

### Example 2

### Fermented Beverages (Alcoholic or Non-Alcoholic)

In certain embodiments, a brewer's wort is prepared which comprises any of a number of ingredients suitable for the preparation of any of a number of fermented beverages. In certain embodiments the fermented beverage is non-alcoholic. Examples of non-alcoholic beverages include, but are not limited to, kombucha, non-alcoholic beer, non-alcoholic wine, fermented tea, sodas, and kefir. The ingredients suitable for the preparation of the wort may vary depending on the non-alcoholic beverage selected, wherein the necessary ingredients for a particular non-alcoholic fermented beverage is known to one of ordinary skill in the art.

In certain embodiments, the wort is inoculated with at least one yeast strain to provide an inoculated wort. In certain embodiments, the yeast strain is GY7B. In certain embodiments, the wort is inoculated with 1.0×10⁶ yeast cells/mL. In certain embodiments, the optimal concentration (i.e. pitch rate or initial cell count) influences lactic acid concentration and/or production. In certain embodiments, the optimal concentration ranges from about 0.5 to about 1.5 g/L (dry yeast) or about 1×10⁶ yeast cells/mL (active yeast in suspension). In certain embodiments, a concentration of dry yeast which is ≤0.5 g/mL or ≥2.5 g/L may result in decreased lactic acid production.

In certain embodiments, the inoculated wort is contained a suitable fermentation vessel. In certain embodiments, the fermentation vessel is at least partially sealed. In certain embodiments, the fermentation vessel is completely sealed. In certain embodiments, fermentation is initiated upon containment of the inoculated wort in the at least partially sealed or completely sealed fermentation vessel.

In certain embodiments, GY7B yeast is added after fermentation has been initiated. The GY7B yeast may be added 1, 2, 3, 4, 5, and/or 6 days after fermentation has been initiated. In certain embodiments, the GY7B yeast is added to the fermentation vessel within 5 days of the initiation of fermentation.

In certain embodiments, any of a number of supplements may be added to decrease the production of ethanol and/or increase the production of lactic acid. In certain embodiments, the supplement is an enzyme. Without wishing to be bound by theory, the enzyme may function to increase the concentration of glucose by the hydrolysis of complex carbohydrates. In certain embodiments, the enzyme may serve to facilitate at least one of an increase in lactic acid production and/or a decrease in ethanol production.

In certain embodiments, the supplement may also comprise a sugar. In certain embodiments, the supplement may comprise a fruit and/or a processed fruit derivative. Non-limiting examples of processed fruit derivatives include fruit puree, macerations, extracts, syrups, juices, and/or dehydrated fruit.

In certain embodiments, the supplement is added after fermentation has been initiated. The supplement may be added 1, 2, 3, 4, 5, and/or 6 days after fermentation has been initiated. In certain embodiments, the supplement is added to the fermentation vessel within 5 days of the initiation of fermentation.

In certain embodiments, the fermentation yields a non-alcoholic beverage. In certain embodiments, the non-alcoholic beverage has an ethanol content of ≤0.5% alcohol (v/v).

In certain embodiments, the production of alcohol during fermentation is reduced or halted after a period of time. In certain embodiments, the reduction of alcohol production is achieved by removal and/or deactivation of at least one yeast strain. In certain embodiments, the removed and/or deactivated yeast strain is GY7B. The yeast strain may be removed and/or deactivated by any of a number of methods. Non-limiting examples of methods of removing at least one yeast strain, including GY7B, includes centrifugation, filtration, and physical separation, which may be facilitated by low temperatures. Non-limiting examples of methods of deactivating at least one yeast strain, including GY7B, includes exposure to low temperature, pasteurization, and/or the addition of chemical inhibitors of fermentation.

### Example 3

### GY7B as a Flocculant

In certain embodiments, GY7B is used as a flocculant to assist with the removal of yeast after fermentation. The alcoholic or non-alcoholic beverage prepared by fermentation may or may not have been fermented utilizing GY7B. GY7B yeast may be added to the fermentation vessel upon completion of the fermentation process, thereby leading to the precipitation of yeast present in the fermented beverage and enabling more effective centrifugation, filtration, pasteurization, and/or any other stabilizing and/or clarification processes performed after fermentation.

### Enumerated Embodiments

The following exemplary embodiments are provided, the numbering of which is not to be construed as designating levels of importance:

Embodiment 1 provides a method of producing sourdough bread, the method comprising baking fermented dough, wherein the fermented dough comprises yeast strain GY7B.

Embodiment 2 provides the method of Embodiment 1, wherein the dough is fermented in the absence of any acid producing bacteria.

Embodiment 3 provides the method of any of Embodiments 1-2, wherein the fermented dough is prepared from a starter culture.

Embodiment 4 provides the method of any of Embodiments 1-3, wherein the starter culture comprises flour, water, and at least one yeast strain.

Embodiment 5 provides the method of Embodiment 4, wherein the at least one yeast strain is yeast strain GY7B.

Embodiment 6 provides the method of any of Embodiments 4-5, wherein the at least one yeast strain selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus Hansensiaspora uvarum Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae Wickerhamomyces subpelliculosus, Wickerhamomyces anomalus, Zygosaccharomyces rouxii, Zygosaccharomyces bailii, Zygosaccharomyces fermentati, Zygosaccharomycesflorentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

Embodiment 7 provides the method of any of Embodiments 1-6, wherein the fermented dough comprises at least one supplement.

Embodiment 8 provides the method of Embodiment 7, wherein the at least one supplement is selected from the group consisting of sugar and yeast extract.

Embodiment 9 provides the method of Embodiment 8, wherein the sugar is selected from the group consisting of glucose, sucrose, fructose, and maltose.

Embodiment 10 provides the method of any of Embodiments 8-9, wherein the yeast extract comprises at least one selected from the group consisting of zinc salts, calcium salts, magnesium salts, lipids, peptides, and amino acids.

Embodiment 11 provides a method of producing a non-alcoholic fermented beverage, the method comprising fermenting a substrate in the presence of yeast strain GY7B.

Embodiment 12 provides the method of Embodiment 11, wherein the substrate is fermented in the absence of any acid producing bacteria.

Embodiment 13 provides the method of any of Embodiments 11-12, wherein the substrate is fermented in the presence of at least one additional yeast strain selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus Hansensiaspora uvarum Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae Wickerhamomyces subpelliculosus, Wickerhamomyces anomalus, Zygosaccharomyces rouxii, Zygosaccharomyces bailii, Zygosaccharomyces fermentati, Zygosaccharomycesflorentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

Embodiment 14 provides the method of any of Embodiments 11-13, wherein the non-alcoholic fermented beverage has a low pH.

Embodiment 15 provides the method of any of Embodiments 11-14, wherein the non-alcoholic fermented beverage has an amount of ethanol ranging from about 0.05% to about 0.5% (v/v).

Embodiment 16 provides the method of any of Embodiments 11-15, wherein the substrate is at least one selected from the group consisting of malt, must, honey, and tea. Embodiment 17 provides the method of any of Embodiments 11-16, wherein the fermentation comprises at least one supplement.

Embodiment 18 provides the method of any of Embodiments 11-18, wherein the at least one supplement is selected from the group consisting of a fruit, processed fruit derivative, and an enzyme.

Embodiment 19 provides the method of Embodiment 18, wherein the fruit or processed fruit derivative is a sugar.

Embodiment 20 provides the method of Embodiment 19, wherein the sugar is glucose.

Embodiment 21 provides the method of Embodiment 20, wherein the glucose is added in an amount ranging from about 1% to about 5% substrate to total fermentation volume (w/v).

Embodiment 22 provides the method of any of Embodiments 18-21, wherein the enzyme is selected from the group consisting of a β-glucosidase and amylase.

Embodiment 23 provides the method of any of Embodiments 17-22, wherein the supplement is present during early fermentation.

Embodiment 24 provides the method of any of Embodiments 11-23, wherein the fermentation occurs with a yeast cell count of about 1.0×10⁶ cells/mL.

Embodiment 25 provides the method of any of Embodiments 11-24, wherein the fermentation occurs with a yeast cell count ranging from about 0.5 g/L to about 1.5 g/L.

Embodiment 26 provides the method of any of Embodiments 11-25, wherein the GY7B yeast strain is removed and/or inactivated.

Embodiment 27 provides the method of Embodiment 26, wherein the removal comprises at least one of centrifugation, filtration, and physical separation.

Embodiment 28 provides the method of any of Embodiments 26-27, wherein the inactivation comprises at least one of low temperature exposure, pasteurization, and chemical inhibitor.

Embodiment 29 provides the method of Embodiment 28, wherein the chemical inhibitor is potassium sorbate.

Embodiment 30 provides a method of promoting flocculation of at least one yeast strain, the method comprising adding yeast strain GY7B to a fermentation vessel comprising the at least one yeast strain before and/or after fermentation of a substrate.

Embodiment 31 provides the method of Embodiment 30, wherein the substrate comprises a malt derived from at least one grain selected from the group consisting of barley, wheat, corn, rye, rice, oats, sorghum, millet, buckwheat, quinoa, and teff.

Embodiment 32 provides the method of any of Embodiments 30-31, wherein the at least one yeast strain is yeast strain GY7B.

Embodiment 33 provides the method of any of Embodiments 30-32, wherein the at least one yeast strain is selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus Hansensiaspora uvarum Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae Wickerhamomyces subpelliculosus, Wickerhamomyces anomalus, Zygosaccharomyces rouxii, Zygosaccharomyces bailii, Zygosaccharomyces fermentati, Zygosaccharomycesflorentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

Embodiment 34 provides a method of producing an alcoholic fermented beverage, the method comprising fermenting a substrate in the presence of yeast strain GY7B, wherein the fermented alcoholic beverage is selected from the group consisting of wine, cider, seltzer, and mead.

Embodiment 35 provides the method of Embodiment 34, wherein the alcoholic fermented beverage is produced without both sequential fermentation and co-fermentation. Embodiment 36 provides the method of any of Embodiments 34-35, wherein the alcoholic fermented beverage has a low pH.

Embodiment 37 provides the method of Embodiment 14 or 36, wherein the pH has a range of about pH 2.0 to about pH 4.0.

Embodiment 38 provides the method of any of Embodiments 34-37, wherein the substrate is fermented in the absence of an acid producing bacteria.

Embodiment 39 provides the method of any of Embodiments 34-38, wherein the fermentation of the substrate occurs in the presence of at least one additional yeast strains selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus Hansensiaspora uvarum Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae Wickerhamomyces subpelliculosus, Wickerhamomyces anomalus, Zygosaccharomyces rouxii, Zygosaccharomyces bailii, Zygosaccharomyces fermentati, Zygosaccharomycesflorentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A method of producing sourdough bread, the method comprising baking fermented dough, wherein the fermented dough comprises yeast strain GY7B.

2. The method of claim 1, wherein at least one of the following applies:
(a) the dough is fermented in the absence of any acid producing bacteria;
(b) the fermented dough is prepared from a starter culture;
(c) the fermented dough comprises at least one supplement.

3. (canceled)

4. The method of claim 2, wherein the fermented dough is prepared from a starter culture, and wherein the starter culture comprises flour, water, and at least one yeast strain.

5. The method of claim 4, wherein at least one of the following applies:
(a) the at least one yeast strain is yeast strain GY7B,
(b) the at least one yeast strain is selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus Hansensiaspora uvarum Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae Wickerhamomyces subpelliculosus, Wickerhamomyces anomalus, Zygosaccharomyces rouxii, Zygosaccharomyces bailii, Zygosaccharomyces fermentati, Zygosaccharomycesflorentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

6-7. (canceled)

8. The method of claim 2, wherein the fermented dough comprises at least one supplement, and wherein the at least one supplement is selected from the group consisting of sugar and yeast extract.

9. The method of claim 8, wherein at least one of the following applies:
(a) the sugar is selected from the group consisting of glucose, sucrose, fructose, and maltose;
(b) the yeast extract comprises at least one selected from the group consisting of zinc salts, calcium salts, magnesium salts, lipids, peptides, and amino acids.

10. (canceled)

11. A method of producing a non-alcoholic fermented beverage, the method comprising fermenting a substrate in the presence of yeast strain GY7B.

12. The method of claim 11, wherein at least one of the following applies:
(a) the substrate is fermented in the absence of any acid producing bacteria;
(b) the substrate is fermented in the presence of at least one additional yeast strain selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus Hansensiaspora uvarum Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae Wickerhamomyces subpelliculosus, Wickerhamomyces anomalus, Zygosaccharomyces rouxii, Zygosaccharomyces bailii, Zygosaccharomyces fermentati, Zygosaccharomycesflorentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala;
(c) the non-alcoholic fermented beverage has a low pH;
(d) the non-alcoholic fermented beverage has an amount of ethanol ranging from about 0.05% to about 0.5% (v/v);
(e) the substrate is at least one selected from the group consisting of malt, must, honey, and tea;
(f) the fermentation comprises at least one supplement;
(g) the fermentation occurs with a yeast cell count of about 1.0×10⁶ cells/mL;
(h) the fermentation occurs with a yeast cell count ranging from about 0.5 g/L to about 1.5 g/L;
(i) the GY7B yeast strain is removed from or inactivated in the non-alcoholic fermented beverage.

13-17. (canceled)

18. The method of claim 12, wherein the fermentation comprises at least one supplement, and wherein the at least one supplement is selected from the group consisting of a fruit, processed fruit derivative, and an enzyme.

19. The method of claim 18, wherein at least one of the following applies:
(a) the fruit or processed fruit derivative comprises a sugar;
(b) the enzyme is selected from the group consisting of a B-glucosidase and amylase.

20. The method of claim 19, wherein the fruit or processed fruit derivative comprises a sugar, and wherein the sugar is glucose.

21. The method of claim 20, wherein the glucose is added in an amount ranging from about 1% to about 5% substrate to total fermentation volume (w/v).

22. (canceled)

23. The method of claim 12, wherein the fermentation comprises a supplement, and wherein the supplement is present during early fermentation.

24-26. (canceled)

27. The method of claim 12, wherein the GY7B yeast strain is removed from or inactivated in the non-alcoholic fermented beverage, and wherein at least one of the following applies:
(a) the GY7B yeast strain is removed from the non-alcoholic fermented beverage using at least one of centrifugation, filtration, and physical separation;
(b) the GY7B yeast strain is inactivated in the non-alcoholic fermented beverage, and the inactivation comprises at least one of low temperature exposure, pasteurization, and chemical inhibitor.

28. (canceled)

29. The method of claim 27, wherein the inactivation comprises a chemical inhibitor, and wherein the chemical inhibitor is potassium sorbate.

30. A method of promoting flocculation of at least one yeast strain, the method comprising adding yeast strain GY7B to a fermentation vessel comprising the at least one yeast strain before and/or or after fermentation of a substrate.

31. The method of claim 30, wherein at least one of the following applies:
(a) the substrate comprises a malt derived from at least one grain selected from the group consisting of barley, wheat, corn, rye, rice, oats, sorghum, millet, buckwheat, quinoa, and teff:
(b) the at least one yeast strain is yeast strain GY7B;
(c) the at least one yeast strain is selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus Hansensiaspora uvarum Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae Wickerhamomyces subpelliculosus, Wickerhamomyces anomalus, Zygosaccharomyces rouxii, Zygosaccharomyces bailii, Zygosaccharomyces fermentati, Zygosaccharomycesflorentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

32-33. (canceled)

34. A method of producing an alcoholic fermented beverage, the method comprising fermenting a substrate in the presence of yeast strain GY7B, wherein the fermented alcoholic beverage is selected from the group consisting of wine, cider, seltzer, and mead.

35. The method of claim 34, wherein at least one of the following applies:
(a) the alcoholic fermented beverage is produced without both sequential fermentation and co-fermentation;
(b) the alcoholic fermented beverage has a low pH;
(c) the substrate is fermented in the absence of an acid producing bacteria;
(d) the fermentation of the substrate occurs in the presence of at least one additional yeast strains selected from the group consisting of Saccharomyces cerevisiae, Saccharomyces pastorianus, Saccharomyces paradoxus, Saccharomyces eubayanus, Saccharomyces ludwigii, Aureobasidium pullulans, Cyberlindnera saturnus Hansensiaspora uvarum Hansensiaspora guilliermondii, Hansensiaspora osmophila, Hansensiasporavineae, Hansenula anomala, Issatchenkia occidentalis, Issatchenkia orientalis, Pichia kluyveri, Pichia caribbica, Pichia fermentans, Pichia kudriavzevii, Pichia Membranifaciens, Rhodotorula mucilaginosa, Torulaspora delbrueckii, Candida colliculosa, Candida shehatae, Candida tropicalis, Candida ethanolica, Candida krusei, Candida magnolia, Candida milleri, Clavispora lusitaniae Wickerhamomyces subpelliculosus, Wickerhamomyces anomalus, Zygosaccharomyces rouxii, Zygosaccharomyces bailii, Zygosaccharomyces fermentati, Zygosaccharomycesflorentinus, Kluyveromyces lactis, Kluyveromyces marxianus, Lachancea thermotolerans, Brettanomyces bruxellensis, Brettanomyces anomalus, Brettanomyces custersianus, Brettanomyces naardenensis, Brettanomyces nanus, Dekkera bruxellensis, and Dekkera anomala.

36. (canceled)

37. The method of claim 35, wherein the alcoholic fermented beverage has a low pH, and wherein the pH has a range of about pH 2.0 to about pH 4.0.

38-39. (canceled)
